(19) United States
(12) Patent Application Publication
(10) Pub. No.: US 2021/0398620 A1
(43) Pub. Date: Dec. 23, 2021

(54) METHODS, SYSTEMS, AND COMPUTER READABLE MEDIA FOR ANALYZING SIMULATED SOLVENT-MEDIATED MOLECULAR INTERACTIONS

(71) Applicant: Arizona Board of Regents on behalf of Arizona State University, Scottsdale, AZ (US)

(72) Inventors: Matthias Heyden, Phoenix, AZ (US); Edgar Manriquez-Sandoval, Baltimore, MD (US)

(73) Assignee: Arizona Board of Regents on behalf of Arizona State University, Scottsdale, AZ (US)

(21) Appl. No.: 17/346,498

(22) Filed: Jun. 14, 2021

Related U.S. Application Data
(60) Provisional application No. 63/039,872, filed on Jun. 16, 2020.

(51) Int. Cl. G16C 20/50; G16C 20/10; G16C 20/80; G16C 10/00

(52) U.S. Cl. CPC G16C 20/50; G16C 10/00; G16C 20/80; G16C 20/10

(57) ABSTRACT

Provided herein are methods of analyzing simulated solvent-mediated molecular interactions. The methods include defining a plurality of three-dimensional (3D) grids of voxels on simulated target molecules solvated with simulated solvent molecules to produce a 3D simulation structure. The simulated solvent molecules include simulated nonionic solvent molecules and simulated ionic solvent molecules. A first 3D grid of the plurality of 3D grids includes a first spatial resolution and is defined on the simulated nonionic solvent molecules. A second 3D grid of the plurality of 3D grids includes a second spatial resolution that differs from the first spatial resolution and is defined on the simulated ionic solvent molecules. Related systems and computer readable media are also provided.

### CROSS-REFERENCE TO RELATED APPLICATIONS

This application claims priority to U.S. Provisional Patent Application Ser. No. 63/039,872, filed Jun. 16, 2020, the disclosure of which is incorporated herein by reference.

### BACKGROUND

Atomistic simulations are computational methods that model materials at the level of atoms and include techniques, such as molecular statics (MS) and molecular dynamics (MD). In the case of MD, for example, computer simulations are used to analyze the physical motion and interaction of atoms and molecules. MD simulations of explicit solvents (the solvent molecules are included in the atomistic representation of the simulated system) intrinsically include solvent-mediated interactions between solvated molecules (solutes), which are a consequence of interactions between the solutes and the solvent and the resulting solvation free energy of the solutes. An accurate theoretical model of solvation free energies and solvent-mediated interactions for complex biomolecular solutes that does not require explicit simulations of the solvent does not yet exist, which is why an explicit simulation of the solvent is often required despite high computational costs involved. In explicit solvent simulations, the number of solvent atoms is typically >90% of the total number of simulated atoms and the computational costs scales at least linearly with the number of atoms in the most efficient software implementations of MD simulations. Theoretical descriptions of thermodynamic properties of the solvent (enthalpy, entropy, free energy) and solute solvation free energies allows for simulations with implicit solvation, in which the solvent molecules do not need to be treated explicitly. In the case of pure water as the solvent, detailed information on the solvent enthalpy, entropy and solvation free energy can be obtained from the analysis of explicit solvent simulations with suitable methods that include the spatially resolved 3D-2PT approach.

To provide models of solvent-mediated molecular interactions that more closely mimic solvation environments observed in vivo, sampling the solvation free energy of electrolytic solvents, which include simulated ions in addition to simulated water molecules, is desired. A major difficulty with including ions in the analysis of thermodynamic properties of solvent molecules and solvation free energies from MD simulations is that the number of simulated ions in the simulated system is typically significantly smaller (e.g., about 150 times smaller) than the number of simulated water molecules. To provide information on the contributions of simulated ions under these conditions with the same spatial resolution as that used for simulated water molecules would involve significantly longer simulations than those involving simulated water molecules alone and are currently essentially infeasible.

Accordingly, there is a need for additional methods, and related aspects, of 3D-2PT analyses that allow for the evaluation of solvation free energy thermodynamics in ionic solutions, that include simulated ionic solvent molecules in addition to simulated nonionic solvent molecules, such as simulated water molecules.

### SUMMARY

The present disclosure relates, in certain aspects, to methods of analyzing solvation free energies in MD simulations with an explicit solvent and predicting solvent-mediated interactions that involve simulated target molecules solvated with simulated solvent solutions that include both simulated ionic and nonionic solvent molecules. To permit computationally feasible simulations, these embodiments also involve the use of hybrid or mixed spatial resolution techniques in which respective ionic and nonionic contributions to solvation free energies are analyzed with 3D grids having different spatial resolutions. These and other aspects will be apparent upon a complete review of the present disclosure, including the accompanying figures.

In one aspect, the present disclosure provides a method of analyzing solvation free energies and predicting solvent-mediated interactions between solvated molecules using a computer. The method includes defining, by the computer, a plurality of three-dimensional (3D) grids of voxels on at least a portion of two or more simulated target molecules solvated with simulated solvent molecules to produce a 3D simulation structure. The simulated solvent molecules comprise one or more simulated nonionic solvent molecules and one or more simulated ionic solvent molecules. A first 3D grid of the plurality of 3D grids comprises a first spatial resolution, which first 3D grid is defined on at least some of the simulated polar solvent molecules. A second 3D grid of the plurality of 3D grids comprises a second spatial resolution that differs from the first spatial resolution, which second 3D grid is defined on at least some of the simulated ionic solvent molecules. The method also includes determining, by the computer, one or more thermodynamic, dynamic, and/or structural parameters using the 3D simulation structure as part of one or more atomistic simulations to produce at least one set of simulation data. In addition, the method also includes generating, by the computer, at least one 3D solvation free energy map from the set of simulation data, thereby analyzing thermodynamic properties of solvent molecules and solvation free energies.

In some embodiments, at least two of the simulated target molecules form a complex with one another. In other embodiments, at least two of the simulated target molecules are separate from one another. In certain embodiments, the methods disclosed herein include determining a difference in solvation free energies between when the simulated target molecules form a complex with one another and when the simulated target molecules are separate from one another. In some embodiments, at least two of the simulated target molecules are identical to one another. In certain embodiments, at least two of the simulated target molecules differ from one another. In some embodiments, the simulated target molecules comprise a simulated biomolecule, a simulated pharmaceutical molecule, a simulated organic molecule, a simulated inorganic molecule, a portion thereof, or a combination thereof.

In certain embodiments, the simulated nonionic solvent molecules comprise simulated water molecules. In some embodiments, the simulated ionic solvent molecules comprise simulated monoatomic ionic molecules. In certain embodiments, a concentration of the simulated nonionic solvent molecules is higher than a concentration of the simulated ionic solvent molecules in the 3D simulation structure.

In some embodiments, the methods disclosed herein further include identifying one or more at least potential binding sites on one or more of the simulated target molecules from the 3D solvation free energy map. In certain embodiments, the methods disclosed herein further include determining an impact of solvation on binding affinity between the simulated target molecules from the 3D solvation free energy map.

In some embodiments, a volume of a given simulated nonionic solvent molecule is greater than a volume of a given voxel in the first 3D grid. In some embodiments, a volume of a given simulated ionic solvent molecule is less than a volume of a given voxel in the second 3D grid. In certain embodiments, the first spatial resolution is higher than the second spatial resolution. Typically, the first spatial resolution and the second spatial resolution are sufficient to obtain substantially continuous statistics from the set of simulation data. In some embodiments, for example, the first spatial resolution is about 1 cubic Angstroms (Å³). In other exemplary embodiments, the second spatial resolution is about 125 cubic Angstroms (Å³).

In some embodiments, the methods disclosed herein include determining spatially resolved enthalpy and entropy contributions from the simulated nonionic solvent molecules and from the simulated ionic solvent molecules. In certain embodiments, the methods disclosed herein include distinguishing interactions between the simulated target molecules and the simulated nonionic solvent molecules, interactions between the simulated target molecules and the simulated ionic solvent molecules, interactions between the simulated nonionic solvent molecules, interactions between the simulated ionic solvent molecules, and interactions between the simulated nonionic solvent molecules and the simulated ionic solvent molecules from one another. In certain embodiments, the methods disclosed herein include sampling interactions between the simulated nonionic solvent molecules and the simulated ionic solvent molecules in both the first and second spatial resolutions.

In other aspects, the present disclosure provides a system that includes at least one controller that comprises, or is capable of accessing, computer readable media comprising non-transitory computer-executable instructions which, when executed by at least one electronic processor, perform at least defining a plurality of three-dimensional (3D) grids of voxels on at least a portion of two or more simulated target molecules solvated with simulated solvent molecules to produce a 3D simulation structure, wherein the simulated solvent molecules comprise one or more simulated nonionic solvent molecules and one or more simulated ionic solvent molecules, wherein a first 3D grid of the plurality of 3D grids comprises a first spatial resolution, which first 3D grid is defined on at least some of the simulated nonionic solvent molecules, and wherein a second 3D grid of the plurality of 3D grids comprises a second spatial resolution that differs from the first spatial resolution, which second 3D grid is defined on at least some of the simulated ionic solvent molecules. The instructions also perform determining one or more thermodynamic, dynamic, and/or structural parameters using the 3D simulation structure as part of one or more atomistic simulations to produce at least one set of simulation data. In addition, the instructions also perform generating at least one 3D solvation free energy map from the set of simulation data.

In still other aspects, the present disclosure provides a computer readable media comprising non-transitory computer-executable instructions which, when executed by at least one electronic processor, perform at least a step of defining a plurality of three-dimensional (3D) grids of voxels on at least a portion of two or more simulated target molecules solvated with simulated solvent molecules to produce a 3D simulation structure, wherein the simulated solvent molecules comprise one or more simulated nonionic solvent molecules and one or more simulated ionic solvent molecules, wherein a first 3D grid of the plurality of 3D grids comprises a first spatial resolution, which first 3D grid is defined on at least some of the simulated nonionic solvent molecules, and wherein a second 3D grid of the plurality of 3D grids comprises a second spatial resolution that differs from the first spatial resolution, which second 3D grid is defined on at least some of the simulated ionic solvent molecules. The instructions also perform determining one or more thermodynamic, dynamic, and/or structural parameters using the 3D simulation structure as part of one or more atomistic simulations to produce at least one set of simulation data. In addition, the instructions also perform generating at least one 3D solvation free energy map from the set of simulation data.

In certain embodiments of the systems and computer readable media disclosed herein, at least two of the simulated target molecules form a complex with one another. In some embodiments of the systems and computer readable media disclosed herein, at least two of the simulated target molecules are separate from one another. In certain embodiments of the systems and computer readable media disclosed herein, the instructions further perform at least determining a difference in solvation free energies between when the simulated target molecules form a complex with one another and when the simulated target molecules are separate from one another. In certain embodiments of the systems and computer readable media disclosed herein, at least two of the simulated target molecules are identical to one another. In some embodiments of the systems and computer readable media disclosed herein, the simulated target molecules comprise a simulated biomolecule, a simulated pharmaceutical molecule, a simulated organic molecule, a simulated inorganic molecule, a portion thereof, or a combination thereof.

In certain embodiments of the systems and computer readable media disclosed herein, the simulated nonionic solvent molecules comprise simulated water molecules. In some embodiments of the systems and computer readable media disclosed herein, the simulated ionic solvent molecules comprise simulated monoatomic ionic molecules. In certain embodiments of the systems and computer readable media disclosed herein, a concentration of the simulated nonionic solvent molecules is higher than a concentration of the simulated ionic solvent molecules in the 3D simulation structure.

In certain embodiments of the systems and computer readable media disclosed herein, the instructions further perform at least identifying one or more at least potential binding sites on one or more of the simulated target molecules from the 3D solvation free energy map. In some embodiments of the systems and computer readable media disclosed herein, the instructions further perform at least determining an impact of solvation on binding affinity between the simulated target molecules from the 3D solvation free energy map.

In certain embodiments of the systems and computer readable media disclosed herein, a volume of a given simulated nonionic solvent molecule is greater than a volume of a given voxel in the first 3D grid. In some embodiments of the systems and computer readable media disclosed herein, a volume of a given simulated ionic solvent molecule is less than a volume of a given voxel in the second 3D grid. In certain embodiments of the systems and computer readable media disclosed herein, the first spatial resolution is higher than the second spatial resolution. In certain embodiments of the systems and computer readable media disclosed herein, the first spatial resolution and the second spatial resolution are sufficient to obtain substantially continuous statistics from the set of simulation data. In some embodiments of the systems and computer readable media disclosed herein, the first spatial resolution is about 1 cubic Angstroms (Å³). In some embodiments of the systems and computer readable media disclosed herein, the second spatial resolution is about 125 cubic Angstroms (Å³).

In certain embodiments of the systems and computer readable media disclosed herein, the instructions further perform at least determining spatially resolved enthalpy and entropy contributions from the simulated nonionic solvent molecules and from the simulated ionic solvent molecules. In some embodiments of the systems and computer readable media disclosed herein, the instructions further perform at least distinguishing interactions between the simulated target molecules and the simulated nonionic solvent molecules, interactions between the simulated target molecules and the simulated ionic solvent molecules, interactions between the simulated nonionic solvent molecules, interactions between the simulated ionic solvent molecules, and interactions between the simulated nonionic solvent molecules and the simulated ionic solvent molecules from one another. In certain embodiments of the systems and computer readable media disclosed herein, the instructions further perform at least sampling interactions between the simulated nonionic solvent molecules and the simulated ionic solvent molecules in both the first and second spatial resolutions.

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically shows exemplary method steps of analyzing solvation free energies and predicting solvent-mediated molecular interaction according to some aspects disclosed herein.

FIG. 2 is a schematic diagram of an exemplary system suitable for use with certain aspects disclosed herein.

FIGS. 3 A and B show plots of entropy contribution (FIG. 3A; vibrational density of states (VDoS) (arb. u.) (y-axis) and wavenumbers (cm⁻¹) (x-axis)) and distribution of entropy information from intermolecular vibrations in a water hydrogen bond (HB) network (FIG. 3B; entropy density (J/(K mol cm⁻¹) (y-axis) and wavenumbers (cm⁻¹) (y-axis)).

FIGS. 4 A and B schematically show different grid resolutions. In particular, as shown the grid volumes containing water (FIG. 4B) cannot experience intra-voxel interactions. This is due to the higher spatial resolution, where voxel volumes are less than water molecule volumes. In contrast, ion grid volumes (FIG. 4A) can experience intravoxel interactions due to the lower spatial resolution. As shown, voxel volumes are large enough to contain multiple monoatomic ion volumes.

FIG. 5 schematically shows pairwise additive interactions between water-ion (wi) and ion-water (iw) molecules.

FIGS. 6A-C show a Lambda Repressor N-Terminal domain (NTD)-DNA complex solvated in both a water and electrolytic solvation environment with a negative 39e⁻ charge. In particular, FIG. 6A shows the total solvation free energy and separated contributions by water and ions at the first hydration shell, approximately 3 Å from the solute surface. The solvent environment includes 39 counter-charge Na⁺ ions and 150 mM NaCl. Ions in the solvent preferentially solvate the charged DNA backbone seen in ΔGᵢ. FIG. 6B shows the solvation free energy of the solute resolved in a pure water solvent. Upon spatial integration, ΔGₛₒₗᵥ=−10,686 kJ/mol similar to the solvation free energy contribution by water in the electrolytic system, ΔGₛₒₗᵥ=−10,504 kJ/mol. FIG. 6C shows the solvation sites for water and ion as number densities 1.3 and 20 times greater than bulk for water and ion, respectively.

FIG. 7 is a plot (kJ/mol (y-axis) and solute complex-solvent (x-axis)) with the spatial integration of the solvation energies shown for the Lambda Repressor-DNA complex in pure water, in Na⁺ and K⁺ charged balance systems, and in 150 mM NaCl and KCl solvents.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Atomistic Simulation: As used herein, “atomistic simulation” refers to a computer simulation that models materials at the level of atoms.

Biomolecule: As used herein, “biomolecule” refers to an organic molecule produced by a living organism. Exemplary biomolecules, include without limitation macromolecules, such as nucleic acids, proteins, peptides, oligomers, carbohydrates, and lipids.

Continuous Statistics: As used herein, “continuous statistics” refers to the collection, analysis, interpretation, and/or presentation of numerical data that involves variables that can take an infinite set of values.

Dynamic Parameter. As used herein, “dynamic parameter” refers to a characteristic element or physical property associated with a rate and/or mechanism of a given chemical reaction or other chemical interaction, such as complex formation or binding between or among molecules, molecular rotations or translation.

Solvation Free Energy Map: As used herein, “solvation free energy map” refers to a map that describes possible conformations of a solvent molecular entity, or the spatial positions of interacting molecules in a system, or parameters and their corresponding free energies that contribute to the solvation free energy (e.g., Gibbs solvation free energy).

Inorganic Molecule: As used herein, “inorganic molecule” refers to a molecule that is composed of elements other than carbon.

Ionic: As used herein, “ionic” in the context of an atom or group of atoms (e.g., a molecule) refers an atom or group of atoms that carry an electrical charge.

Monoatomic: As used herein, “monoatomic” refers to substances that are single atoms not covalently bonded to other atoms.

Nonionic: As used herein, “nonionic” in the context of a given molecule refers to a molecule having a non-zero total charge.

Nucleic Acid: As used herein, “nucleic acid” refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., bromodeoxyuridine (BrdU)), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA, cfDNA, ctDNA, or any combination thereof.

Organic Molecule: As used herein, “organic molecule” refers to a molecule that is typically found in or produced by living systems. Organic molecules often include carbon atoms in the form of rings or chains having other attached atoms, such as hydrogen, oxygen, sulfur, phosphorous, and/or nitrogen. Exemplary organic molecules, include nucleic acids, proteins, carbohydrates, and lipids.

Pharmaceutical Molecule: As used herein, “pharmaceutical molecule” refers to a molecule that treats, is suspected of treating, or is a candidate for treating, a disease, condition, or disorder upon being administered to a subject afflicted by the disease, condition, or disorder.

Protein: As used herein, “protein” or “polypeptide” refers to a polymer of at least two amino acids attached to one another by a peptide bond. Examples of proteins include enzymes, hormones, antibodies, and fragments thereof.

Simulated: As used herein, “simulated” or “simulation” in the context of mathematical modeling refers to a process performed using a computer to predict the action or outcome of a real-world physical interaction or system. For example, simulated molecules (e.g., simulated target molecules, simulated nonionic solvent molecules, simulated ionic solvent molecules, and/or the like) can be used to predict the real-world interaction (e.g., binding, complex formation, or other association) between or among those molecules. A “simulation structure” is a construct that includes one or more simulated target molecules solvated with simulated solvent molecules.

Solute: As used herein, “solute” refers to one or more dissolved ions and/or molecules.

Solvation: As used herein, “solvation” refers to the process by which solvent molecules surround and interact with solute ions or molecules.

Solvent: As used herein, “solvent” refers to ions or molecules that are capable of dissolving or dispersing one or more other ions or molecules (i.e., solutes).

Solvent-Mediated Molecular Interaction: As used herein, “solvent-mediated molecular interaction” refers to indirect intermolecular interactions between solute molecules that occur in a given solvent medium as a consequence of the interactions between the solutes and the solvent.

Spatial Resolution: As used herein, “spatial resolution” refers to the number of pixels or voxels utilized in the construction of a digital image or model. Images or models having higher spatial resolution are composed with a greater number of pixels or voxels than those of lower spatial resolution.

Structural Parameter: As used herein, “structural parameter” refers to a characteristic element or physical property associated with a spatial structure or configuration of a molecule or groups of atoms and/or molecules.

Thermodynamic Parameter: As used herein, “thermodynamic parameter” refers to a characteristic element or physical property associated with a relationship between forms of energy, such as heat, mechanical, electrical, or chemical energy.

Voxel: As used herein, “voxel” refers to unit of graphic information that defines a region in three-dimensional space. A “cubic voxel” comprises x, y, and z coordinates of identical edge length to one another that define a cubic region in three-dimensional space. A “non-cubic voxel” comprises x, y, and z coordinates with edge lengths that define a non-cubic region in three-dimensional space.

### DETAILED DESCRIPTION

Solvation free energy drives equilibrium properties of solvent-mediated molecular interactions, and accordingly plays a major role in predictive theoretical models. In biomolecular systems, for example, the prediction of binding free energies for protein-ligand complexes or protein-protein interactions is central to many computational drug design applications. Accurate predictions, however, are often challenging to obtain due to various compensating entropic and enthalpic contributions to the free energy. The difficulties in making these predictions are further compounded when in vivo environments are simulated, such as those in which solutes are solvated with electrolytic solvents that include water molecules at much higher numbers than ionic solvent molecules. In some embodiments, the present disclosure presents hybrid or mixed spatial resolution methods and related aspects that minimize these difficulties when modeling electrolytic solvent systems, among many other attributes.

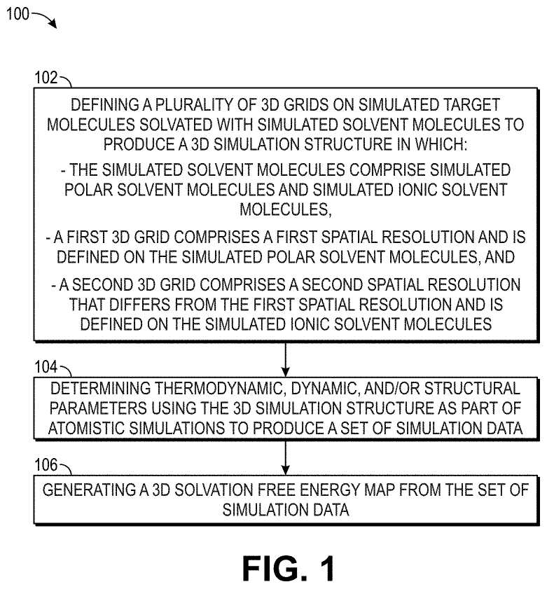

To illustrate, FIG. 1 is a flow chart that schematically shows exemplary method steps of analyzing a simulated solvent-mediated molecular interaction according to some embodiments. As shown, method 100 includes defining a plurality of three-dimensional (3D) grids of voxels on simulated target molecules solvated with simulated solvent molecules to produce a 3D simulation structure (step 102). Essentially any simulated target molecule can be analyzed using method 100. In some embodiments, for example, the simulated target molecules include a simulated biomolecule, a simulated pharmaceutical molecule, a simulated organic molecule, a simulated inorganic molecule, a portion thereof, or a combination thereof. The simulated solvent molecules include simulated nonionic solvent molecules and simulated ionic solvent molecules (e.g., monoatomic molecules, such Na⁺, K⁺, Cl⁻, F⁻, Br⁻, I⁻, or the like). Typically, the simulated nonionic solvent molecules are simulated water molecules, although other simulated nonionic solvent molecules are optionally used. A concentration or number of the simulated nonionic solvent molecules is generally higher than a concentration or number of the simulated ionic solvent molecules in the 3D simulation structure. A first 3D grid of the plurality of 3D grids includes a first spatial resolution and is defined on at least some of the simulated nonionic solvent molecules. In addition, a second 3D grid of the plurality of 3D grids includes a second spatial resolution that differs from the first spatial resolution and is defined on at least some of the simulated ionic solvent molecules.

Method 100 also includes determining thermodynamic, dynamic, and/or structural parameters using the 3D simulation structure as part of one or more atomistic simulations to produce a set of simulation data (step 104). Typically, this includes spatially resolving enthalpy and entropy contributions. Exemplary equations that can be used to determine these spatial resolutions are described in the Example provided herein. Method 100 additionally includes generating a 3D solvation free energy map from the set of simulation data. In some implementations, these simulations are performed using Gromacs software [Abraham et al., SoftwareX, 1-2:19-25 (2015), Páll et al., Proc. of EASC 2015 LNCS, 8759:3-27 (2015), and Pronk et al., Bioinformatics, 29:845-854 (2013)]. Additional software and related systems for performing the methods of the present disclosure are described further herein.

In some embodiments, simulated target molecules at least initially form a complex with one another in the 3D simulation structure. In other embodiments, simulated target molecules are at least initially separate from one another in the 3D simulation structure. In some of these embodiments, for example, method 100 includes determining a difference in solvation free energies between when the simulated target molecules form a complex with one another and when the simulated target molecules are separate from one another. In some embodiments, the simulated target molecules are identical to one another (e.g. two identical polypeptides of a multi-unit enzyme or the like). In other embodiments, the simulated target molecules differ from one another (e.g., an antibody-antigen pair, an enzyme-substrate pair, or the like).

Typically, the 3D grids are composed of cubic voxels, although non-cubic voxels are used in performing certain simulations. In some embodiments, the volume of a given simulated nonionic solvent molecule is greater than the volume of a given voxel in the first 3D grid such that grid volumes containing the simulated nonionic solvent molecule cannot experience intra-voxel interactions. By contrast, in some simulations, the volume of a given simulated ionic solvent molecule is less than the volume of a given voxel in the second 3D grid such that grid volumes containing the simulated ionic solvent molecules can experience intra-voxel interactions (e.g., when multiple simulated ionic solvent molecules are present together in a given voxel) due to the lower spatial resolution in these embodiments. Typically, the spatial resolution of the first 3D grid is higher than the spatial resolution of the second 3D grid. The spatial resolutions of the first and second 3D grids are generally selected so as to obtain substantially continuous statistics from the set of simulation data. Although other voxel edge lengths are optionally used, in some embodiments, the first spatial resolution is about 1 cubic Angstroms (Å³) (i.e., form a 1×1×1 Å³ grid), while the second spatial resolution is about 125 cubic Angstroms (Å³) (i.e., form a 5×5×5 Å³ grid).

Method 100 generally includes determining spatially resolved enthalpy and entropy contributions from the simulated nonionic solvent molecules and from the simulated ionic solvent molecules. In some of these embodiments, method 100 includes distinguishing interactions between the simulated target molecules and the simulated nonionic solvent molecules, interactions between the simulated target molecules and the simulated ionic solvent molecules, interactions between the simulated nonionic solvent molecules, interactions between the simulated ionic solvent molecules, and interactions between the simulated nonionic solvent molecules and the simulated ionic solvent molecules from one another to facilitate the solvation analysis. Method 100 also typically includes sampling interactions between the simulated nonionic solvent molecules and the simulated ionic solvent molecules in both the first and second spatial resolutions, for example, to conserve continuous statistics.

The simulation data and 3D solvation free energy maps generated by method 100 have many different uses. To illustrate, in some embodiments, method 100 also includes identifying potential binding sites on the simulated target molecules from the simulation data and/or 3D solvation free energy map, for example, a part of a drug design process. In other exemplary embodiments, method 100 further includes determining an impact of solvation on binding affinity between the simulated target molecules from the simulation data and/or 3D solvation free energy map, for example, in an effort to improve the efficacy of a given pharmaceutical molecule under evaluation.

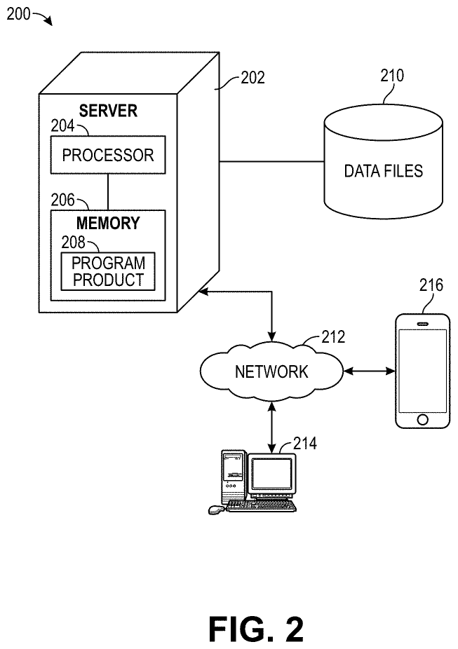

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 2 provides a schematic diagram of an exemplary system suitable for use with implementing at least aspects of the methods disclosed in this application. As shown, system 200 includes at least one controller or computer, e.g., server 202 (e.g., a search engine server), which includes processor 204 and memory, storage device, or memory component 206, and one or more other communication devices 214, 216, (e.g., client-side computer terminals, telephones, tablets, laptops, other mobile devices, etc. (e.g., for receiving simulation data and/or 3D solvation free energy maps for further analysis, etc.) in communication with the remote server 202, through electronic communication network 212, such as the Internet or other internetwork. Communication devices 214, 216 typically include an electronic display (e.g., an internet enabled computer or the like) in communication with, e.g., server 202 computer over network 212 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 200 also includes program product 208 (e.g., for analyzing simulated solvent-mediated molecular interactions as described herein) stored on a computer or machine readable medium, such as, for example, one or more of various types of memory, such as memory 206 of server 202, that is readable by the server 202, to facilitate, for example, a guided search application or other executable by one or more other communication devices, such as 214 (schematically shown as a desktop or personal computer). In some aspects, system 200 optionally also includes at least one database server, such as, for example, server 210 associated with an online website having data stored thereon (e.g., entries corresponding to simulation data, 3D solvation free energy maps, etc.) searchable either directly or through search engine server 202. System 200 optionally also includes one or more other servers positioned remotely from server 202, each of which are optionally associated with one or more database servers 210 located remotely or located local to each of the other servers. The other servers can beneficially provide service to geographically remote users and enhance geographically distributed operations.

As understood by those of ordinary skill in the art, memory 206 of the server 202 optionally includes volatile and/or nonvolatile memory including, for example, RAM, ROM, and magnetic or optical disks, among others. It is also understood by those of ordinary skill in the art that although illustrated as a single server, the illustrated configuration of server 202 is given only by way of example and that other types of servers or computers configured according to various other methodologies or architectures can also be used. Server 202 shown schematically in FIG. 2, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 200. As also understood by those of ordinary skill in the art, other user communication devices 214, 216 in these aspects, for example, can be a laptop, desktop, tablet, personal digital assistant (PDA), cell phone, server, or other types of computers. As known and understood by those of ordinary skill in the art, network 212 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 208 is optionally in the form of microcode, programs, cloud computing format, routines, and/or symbolic languages that provide one or more sets of ordered operations that control the functioning of the hardware and direct its operation. Program product 208, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 208 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 208 is optionally copied from the computer-readable medium to a hard disk or a similar intermediate storage medium. When program product 208, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to provide information that causes at least one 3D solvation free energy map and/or the like to be displayed (e.g., via communication devices 214, 216 or the like) and/or receive information from other system components and/or from a system user (e.g., via communication devices 214, 216, or the like).

In some aspects, program product 208 includes non-transitory computer-executable instructions which, when executed by electronic processor 204 perform at least: defining a plurality of three-dimensional (3D) grids of voxels on at least a portion of two or more simulated target molecules solvated with simulated solvent molecules to produce a 3D simulation structure, wherein the simulated solvent molecules comprise one or more simulated nonionic solvent molecules and one or more simulated ionic solvent molecules, wherein a first 3D grid of the plurality of 3D grids comprises a first spatial resolution, which first 3D grid is defined on at least some of the simulated nonionic solvent molecules, and wherein a second 3D grid of the plurality of 3D grids comprises a second spatial resolution that differs from the first spatial resolution, which second 3D grid is defined on at least some of the simulated ionic solvent molecules. The instructions also perform determining one or more thermodynamic, dynamic, and/or structural parameters using the 3D simulation structure as part of one or more atomistic simulations to produce at least one set of simulation data. In addition, the instructions also perform generating at least one 3D solvation free energy map from the set of simulation data. Other exemplary executable instructions that are optionally performed are described further herein.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), which are each incorporated by reference in their entirety.

### Example

Sampling Solvation Free Energy of Electrolytic Solvents with Three-Dimensional Two-Phase Thermodynamics (3D-2PT)

Interactions between biomolecules and their solvation environment significantly contribute to their stability, driving forces for complex formation, and conformational free energy landscape. In this example, the 3D-2PT method for pure water solvents was expanded to include contributions from electrolytes to the solvation enthalpy, entropy, and free energy from equilibrium molecular dynamic simulations in explicit solvent. This new method is highly suitable for solvation analysis of charges of biomolecules in the presence of counter-charges, as solute-water, solute-ion, water-water, ion-ion and water-ion interactions are now distinguished from one another and analyzed on multiple grids with distinct resolutions.

Solvation free energy calculations included interactions between solvent-solute and solvent-solvent in an electrolytic solvent. Contributions by ions and water were sampled at different spatial resolutions to conserve continuous statistics. Only water-ion interactions were sampled in both resolutions and contributed equally to ion solvation enthalpy and water solvation enthalpy.

Equations used as part of this example included:

$$\begin{matrix} Δ {G}_{solv} = Δ {G}_{W} + Δ {G}_{I} = Δ {H}_{W} + Δ {H}_{I} - T Δ {S}_{W} - T Δ {S}_{I} , & ( I ) \\ = \int Δ {H}_{W} ( r ) \cdot {n}_{W} ( r ) dr + \int Δ {H}_{I} ( r ) \cdot {n}_{I} ( r ) dr - T \int Δ {S}_{W} ( r ) \cdot {n}_{W} ( r ) dr - T \int Δ {S}_{I} ( r ) \cdot {n}_{W} ( r ) dr , & ( II ) \\ Δ {H}_{W} ( r ) = {U}_{SW} ( r ) + Δ {U}_{WI} ( r ) + Δ {U}_{WW} ( r ) , & ( III ) \\ Δ {U}_{WI} ( r ) = \frac{1}{2} ( {U}_{WI} ( r ) - {U}_{WI}^{bulk} ) , & ( IV ) \\ Δ {U}_{WW} ( r ) = ( {U}_{WW}^{{ }^{′}} ( r ) - {U}_{WW}^{bulk {,}^{′}} ) + \frac{1}{2} ( {U}_{WW}^{{ }^{″}} ( r ) - {U}_{WW}^{bulk {,}^{″}} ) , & ( V ) \\ Δ {H}_{I} ( r ) = {U}_{SI} ( r ) + Δ {U}_{IW} ( r ) + Δ {U}_{II} ( r ) , & ( VI ) \\ Δ {U}_{IW} ( r ) = \frac{1}{2} ( {U}_{IW} ( r ) - {U}_{IW}^{bulk} ) , & ( VII ) \\ Δ {U}_{II} ( r ) = ( {U}_{II}^{{ }^{′}} ( r ) - {U}_{II}^{bulk {,}^{′}} ) + \frac{1}{2} ( {U}_{II}^{{ }^{″}} ( r ) - {U}_{II}^{bulk {,}^{″}} ) , & ( VIII ) \\ Δ {S}_{W} ( r ) = {S}_{W} ( r ) - {S}_{W}^{bulk} , & ( IX ) \\ Δ {S}_{I} ( r ) = \frac{{\sum }_{j} ( {S}_{j} ( r ) - {S}_{j}^{bulk} ) \cdot {n}_{j} ( r )}{{\sum }_{j} {n}_{j} ( r )} , & ( X )\end{matrix}$$

where ΔGₛₒₗᵥ is the total solvation free energy of the solute; ΔG_{W} is the contribution to the total solvation free energy from water solvent molecules; ΔG_{I} is the contribution to the total solvation free energy contribution from ionic solvent molecules; ΔH_{W} is the contribution to the total solvation enthalpy from water solvent molecules; ΔH_{I} is the contribution to the total solvation enthalpy from ionic solvent molecules; T is the simulation temperature; ΔS_{W} is the contribution to the total solvation entropy from water solvent molecules; ΔS_{I} is the contribution to the total solvation entropy from ionic solvent molecules; ΔH_{W}(r) describes a local contribution of water solvent molecules (per water molecule) to the solvation enthalpy at position r in the solute environment; n_{W}(r) is the local number density of water molecules at position r in the solute environment; ΔH_{I}(r) describes a local contribution of ionic solvent molecules (per ionic solvent molecule) to the solvation enthalpy at position r in the solute environment; n_{I}(r) is the local number density of ionic solvent molecules at position r in the solute environment; ΔS_{W}(r) describes a local contribution of water solvent molecules (per water molecule) to the solvation enthalpy at position r in the solute environment; ΔS_{I}(r) describes a local contribution of ionic solvent molecules (per ionic solvent molecule) to the solvation enthalpy at position r in the solute environment; dr describes an integration volume element which for numerical integrations over an analysis grid is equivalent to the volume of a single voxel of this grid; U_{SW}(r) describes interactions between the solute molecule and water solvent molecules (per water molecule) at position r in the solute environment; ΔU_{WI}(r) describes the difference of interactions between water solvent molecules at position r in the solute environment and ionic solvent molecules (per water molecule), U_{WI}(r), from the corresponding average of such interactions in the bulk solution, U_{WI}^{bulk}; ΔU_{WW}′(r) describes the difference of interactions between water solvent molecules within the same voxel (intra-voxel) at position r in the solute environment (per water molecule), U_{WW}′(r), from the corresponding of average of such interactions in the bulk solution, U_{WW}^{bulk}′; ΔU_{WW}″(r) describes the difference of interactions between water solvent molecules at position r in the solute environment and all other water solvent molecules (inter-voxel, per water molecule), U_{WW}″(r), from the corresponding of average of such interactions in the bulk solution, U_{WW}^{bulk}″; U_{SI}(r) describes interactions between the solute molecule and ionic solvent molecules (per ionic solvent molecule) at position r in the solute environment; ΔU_{IW}(r) describes the difference of interactions between ionic solvent molecules at position r in the solute environment and water solvent molecules (per ionic solvent molecule), U_{IW}(r), from the corresponding average of such interactions in the bulk solution, U_{IW}^{bulk}; ΔU_{II}′(r) describes the difference of interactions between ionic solvent molecules within the same voxel (intra-voxel) at position r in the solute environment (per ionic solvent molecule), U_{II}′(r), from the corresponding of average of such interactions in the bulk solution, U_{II}^{bulk}″; ΔU_{II}″(r) describes the difference of interactions between ionic solvent molecules at position r in the solute environment and all other ionic solvent molecules (inter-voxel, per ionic solvent molecule), U_{II}″(r), from the corresponding of average of such interactions in the bulk solution, U_{II}^{bulk}″; ΔS_{W}(r) is the difference of the entropy of water solvent molecules at position r in the solute environment (per water molecule), S_{W}(r), from the corresponding average of the entropy per water solvent molecule in the bulk solution, S_{W}^{bulk}; ΔS_{I}(r) is the weighted average difference of the entropy of ionic solvent molecule species j at position r in the solute environment (per ionic solvent molecule of species j), Sⱼ(r), from the corresponding average of the entropy per ionic solvent molecule species j in the bulk solution, Sⱼ^{bulk}, where the local number density of the ionic solvent molecule species j, nⱼ(r), is used as the weighting factor to obtain ΔS_{I}(r) per ionic solvent molecule irrespective of the species ionic solvent molecule species.

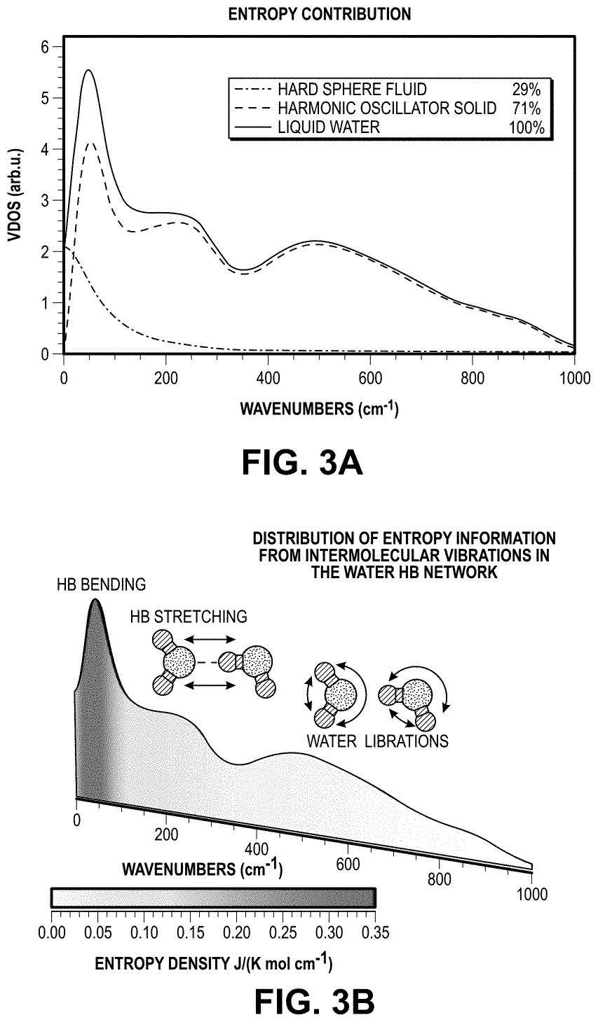

The local entropy of water and ion molecules was determined from the vibrational density of states (VDoS) obtained from Fourier transformed fluctuations of atomic velocities (FIG. 3A). Thermodynamic information was obtained within the 2PT framework, which relates the VDoS of translational and rotational degrees of freedom to analytical models (FIG. 3B).

Spatially Resolved Enthalpy Contributions

Since drastic differences in concentration between water (55M) and ions (0.15M) in electrolytic solutions were present, a mixed resolution approach to spatially resolve the solvation enthalpy was utilized. Lower concentration solvents needed lower spatial resolution to obtain equivalent statistics per voxel. In addition, inter- and intra-grid interactions occurred at lower resolutions.

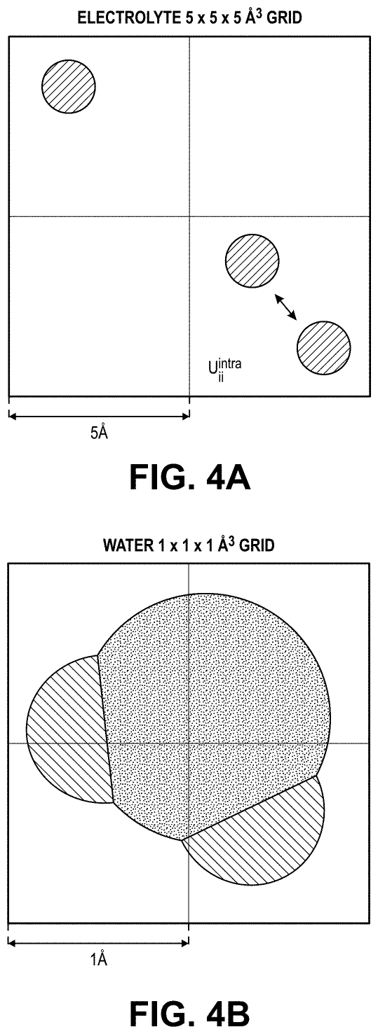

FIGS. 4 A and B schematically show different grid resolutions. In particular, as shown the grid volumes containing water (FIG. 4B) could not experience intra-voxel interactions. This was due to the higher spatial resolution, where voxel volumes were less than water molecule volumes. In contrast, ion grid volumes (FIG. 4A) could experience intravoxel interactions due to the lower spatial resolution. As shown, voxel volumes were large enough to contain multiple monoatomic ion volumes.

Solute-Water Interactions were Determined Using the Following Equations:

$$\begin{matrix} {U}_{SW} ( r ) = 〈 {\sum }_{i ∈ r} {U}_{SW}^{i} 〉 / 〈 {\sum }_{i ∈ r} 1 〉 , & ( XI ) \\ {U}_{SW}^{i} = {\sum }_{a} 4 {ɛ}_{LJ}^{aO} [ {( \frac{{σ}_{LJ}^{aO}}{{r}_{aO}^{i}} )}^{12} - {( \frac{{σ}_{LJ}^{aO}}{{r}_{aO}^{i}} )}^{6} ] + \frac{1}{4 {πɛ}_{0}} [ {\sum }_{a} \sum {b}_{{∈}_{\{ O , {H}_{1} , {H}_{2} \}}} ( \frac{{q}_{a} \cdot {q}_{b}}{{r}_{ab}^{i}} ) ] , & ( XII )\end{matrix}$$

where U_{SW}ⁱ is the interaction energy between the solute and water molecule i; i∈r describes the set of water molecules within the volume of the voxel located at position r in the environment of the solute; a represents an index running over each atom of the solute molecule with partial charge qₐ, ε_{LJ}^{aO} and σ_{LJ}^{aO} are parameters of the Lennard-Jones potential between atom a of the solute and oxygens of both water molecules and r_{aO}ⁱ describes the distance between the coordinates of atom a of the solute and the oxygen of water molecule i; b∈{0, H₁, H₂} indicates the set of oxygen and hydrogen atoms in water molecule i with partial charge q_{b}; the distance between atom a of the solute and atom b of water molecule i is given as r_{ab}ⁱ; [symbol] [symbol] denotes averaging over all time frames of the MD simulation.

Solute-Ion Interactions were Determined Using the Following Equations:

$$\begin{matrix} {U}_{SI} ( r ) = 〈 {\sum }_{j ∈ r} {U}_{SI}^{j} 〉 / 〈 {\sum }_{j ∈ r} 1 〉 , & ( XIII ) \\ {U}_{SI}^{j} = {\sum }_{a} 4 {ɛ}_{LJ}^{aj} [ {( \frac{{σ}_{LJ}^{aj}}{{r}_{a}^{j}} )}^{12} - {( \frac{{σ}_{LJ}^{aj}}{{r}_{a}^{j}} )}^{6} ] + \frac{1}{4 {πɛ}_{0}} [ {\sum }_{a} \frac{{q}_{a} \cdot {q}^{j}}{{r}_{a}^{j}} ] , & ( XIV )\end{matrix}$$

where U_{SW}^{j} is the interaction energy between the solute and ionic solvent molecule j, j∈r describes the set of ionic solvent molecules within the volume of the voxel located at position r in the environment of the solute; a represent an index running over each atom of the solute molecule with partial charge qₐ, ε_{LJ}^{aj} and σ_{LJ}^{aj} are parameters of the Lennard-Jones potential between atom a of the solute and the ionic solvent molecule j, and rₐ^{j} describes the distance between the coordinates of atom a of the solute and the ionic solvent molecule j with partial charge q_{b}; the distance between atom a of the solute and the ionic solvent molecule j is given as rₐ^{j}; [symbol] [symbol] denotes averaging over all time frames of the MD simulation.

Water Inter-Voxel Interactions were Determined Using the Following Equations:

$$\begin{matrix} {U}^{{ }_{WW}^{″}} ( r ) = 〈 {\sum }_{i ∈ r} {\sum }_{j ∉ r} {U}_{WW}^{ij} 〉 / 〈 {\sum }_{i ∈ r} 1 〉 , & ( XV ) \\ {U}_{WW}^{ij} = 4 {ɛ}_{LJ}^{OO} [ {( \frac{{σ}_{LJ}^{OO}}{{r}_{OO}^{ij}} )}^{12} - {( \frac{{σ}_{LJ}^{OO}}{{r}_{OO}^{ij}} )}^{6} ] + \frac{1}{4 {πɛ}_{0}} [ \sum {a}_{{∈}_{\{ O , {H}_{1} , {H}_{2} \}}} \sum {b}_{{∈}_{\{ O , {H}_{1} , {H}_{2} \}}} ( \frac{{q}_{a} \cdot {q}_{b}}{{r}_{ab}^{ij}} ) ] , & ( XVI )\end{matrix}$$

where U_{WW}^{ij} is the interaction energy between water molecules i and j; i∈r describes the set of water molecules within the volume of the voxel located at position r in the environment of the solute and j∉r describes the set of all other water molecules in the system; ε_{LJ}^{OO} and σ_{LJ}^{OO} are parameters of the Lennard-Jones potential between the oxygens of both water molecules and r_{OO}^{ij} describes the distance between the coordinates of the oxygen atoms in water molecules i and j; a, b∈{O, H₁, H₂} indicates the set of oxygen and hydrogen atoms in water molecules i and j with the partial charge qₐ and q_{b} and the distance between the corresponding atoms r_{ab}^{ij}; [symbol] [symbol] denotes averaging over all time frames of the MD simulation.

Electrolyte Intra-Voxel Interactions were Determined Using the Following Equations:

$$\begin{matrix}{U}_{II}^{{ }^{′}} ( r ) = 〈 {\sum }_{i ∈ r} {\sum }_{( j > i ) ∈ r} {U}_{II}^{ij} 〉 / 〈 {\sum }_{i ∈ r} 1 〉 , & ( XVII ) \\ {U}_{II}^{ij} = 4 {ɛ}_{LJ}^{ij} [ {( \frac{{σ}_{LJ}^{ij}}{{r}^{ij}} )}^{12} - {( \frac{{σ}_{LJ}^{ij}}{{r}^{ij}} )}^{6} ] + \frac{1}{4 {πɛ}_{0}} \frac{{q}^{i} \cdot {q}^{j}}{{r}^{ij}} , & ( XVIII )\end{matrix}$$

where U_{II}^{ij} is the interaction energy between the two ions i and j that are located within in the volume of same voxel at location r in the environment of the solute; i∈r describes the set of ionic solvent molecules within the volume of the voxel located at position r in the environment of the solute and j∉r describes the set of all other ionic solvent molecules in the system; ε_{LJ}^{ij} and σ_{LJ}^{ij} are parameters of the Lennard-Jones potential between the ions i and j, qⁱ and q^{j} are the charges of the two ions i and j, r^{ij} is the distance between them and ε₀ is the electric permittivity of the vacuum; [symbol] [symbol] denotes averaging over all time frames of the MD simulation.

Electrolyte inter-voxel interactions were determined using the following Equation:

U_{II}″(r)=(Σ_{i∈r}Σ_{j∉r}U_{II}^{ij})/(Σ_{i∈r}1) (XIX),

where U_{II}^{ij} is defined as in Equation XIV, i∈r describes the set of ions located within the volume of the voxel at position r in the environment of the solute and j∉r describes the set of all other ions in the simulated system.

Ion-water/water-ion interactions were determined using the following Equations:

$$\begin{matrix} {U}_{WI}^{{ }^{″}} ( r ) = 〈 {\sum }_{i ∈ r} {\sum }_{j} {U}_{WI}^{ij} 〉 / 〈 {\sum }_{i ∈ r} 1 〉 , & ( XX ) \\ {U}_{IW}^{{ }^{″}} ( r ) = 〈 {\sum }_{j ∈ r} {\sum }_{i} {U}_{WI}^{ij} 〉 / 〈 {\sum }_{j ∈ r} 1 〉 , & ( XXI ) \\ {U}_{WI}^{ij} = 4 {ɛ}_{LJ}^{Oj} [ {( \frac{{σ}_{LJ}^{OJ}}{{r}_{O}^{ij}} )}^{12} - {( \frac{{σ}_{LJ}^{Oj}}{{r}_{O}^{ij}} )}^{6} ] + \frac{1}{4 {πɛ}_{0}} [ \sum {a}_{{∈}_{\{ O , {H}_{1} , {H}_{2} \}}} ( \frac{{q}_{a} \cdot {q}^{j}}{{r}_{a}^{ij}} ) ] , & ( XXII )\end{matrix}$$

where U_{WI}^{ij} is the interaction energy between water molecule i and ionic solvent molecule j; i∈r describes the set of water molecules within the volume of the voxel located at position r in the environment of the solute, j∈r describes the set of ionic solvent molecules within the volume of the voxel located at position r in the environment of the solute; ε_{LJ}^{Oj} and σ_{LJ}^{Oj} are parameters of the Lennard-Jones potential between the oxygen atom of a water molecule and the ionic solvent molecule j; a, b∈{O, H₁, H₂} indicates the set of oxygen and hydrogen atoms in water molecule i with the partial charges qₐ, q^{j} is the charge of the ionic solvent molecule j, and the distance between the corresponding atoms is rₐ^{ij}; [symbol] [symbol] denotes averaging over all time frames of the MD simulation.

All water-water interactions occurred inter-voxel, i.e. across different voxel units on the grid used to resolve contributions of water to the solvation free energy. The factor ½ in Equation V compensates for double counting during spatial integration. The same applies to inter-voxel interactions between ions as well, which results in the corresponding ½ factor in Equation VIII. Interactions between water molecules and ions are analyzed in terms of water-ion and ion-water interactions and therefore also double counted, which is considered by the corresponding % factors in Equations IV and VII.

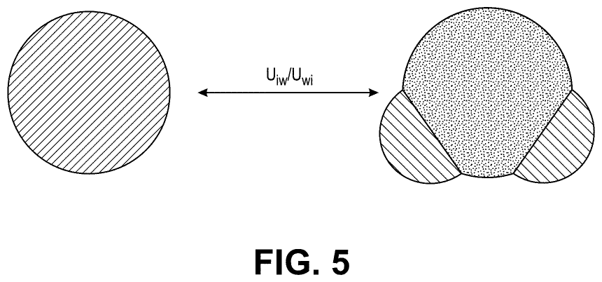

The pair-wise additive interactions between water-ion and ion-water interactions were sampled in both high water grid resolution and low ion grid resolution (FIG. 5). Upon integration, the total contribution was equally attributed to water and ion solvation enthalpies (see above).

Spatially Resolved Entropy Contributions

Solvation entropies were resolved from the vibrations density of states (VDoS) of translational and rotational degrees of freedom (DOF), I_{trans/rot}(v). The VDoS were obtained by analyzing the frequency space of the velocity time autocorrelation function, Cᵥ(τ) for translational velocities v and C_{ω}(τ) for rotational velocities w, tracked over 200 time steps. Water possesses three translational and three rotational DOF, while monoatomic ions possess three translational DOF per ion species. Entropy was then described in terms of the weighted sum of integrals of the VDoS for distinct degrees of freedom as described in 2PT theory [Persson et al., J Chem Theory Comput 13:4467-4481 (2017); Lin et al., J Phys Chem B 114:8191-8198 (2010)].

The spatially resolved entropy contributions were determined using the following Equations:

$$\begin{matrix}{S}_{W} = {S}_{trans}^{HS} + {S}_{trans}^{HO} + {S}_{rot}^{RR} + {S}_{rot}^{HO} = \int {W}_{trans}^{HS} \cdot {I}_{trans}^{HS} ( v ) dv + \int {W}_{trans}^{HS} ( v ) \cdot {I}_{trans}^{HO} ( v ) dv + \int {W}_{rot}^{RR} \cdot {I}_{rot}^{HS} ( v ) dv + \int {W}_{rot}^{HO} ( v ) \cdot {I}_{rot}^{HO} ( v ) dv , & ( XXIII ) \\ {S}_{I} = {S}_{trans}^{HS} + {S}_{trans}^{HO} = \int {W}^{HS} ( v ) \cdot {I}_{trans}^{HS} ( v ) dv + \int {W}^{HO} ( v ) \cdot {I}_{trans}^{HO} ( v ) dv , & ( XXIV ) \\ \frac{2}{{k}_{B} T} [ {C}_{v} ] = {I}_{trans}^{HS} ( v ) + {I}_{trans}^{HO} ( v ) & ( XXV ) \\ \frac{2}{{k}_{B} T} [ {C}_{ω} ] = {I}_{rot}^{HS} ( v ) + {I}_{rot}^{HO} ( v ) , & ( XXVI )\end{matrix}$$

where Sₜᵣₐₙₛ^{HS}, is the hard sphere (HS) entropy contribution of translational degrees of freedom, Sₜᵣₐₙₛ^{HO} is the harmonic oscillator (HO) contribution of translational degrees of freedom, Sᵣₒₜ^{RR} is the rigid rotor (RR) contribution of the rotational degrees of freedom and Sᵣₒₜ^{HO} is the HO contribution of rotational degrees of freedom. Wₜᵣₐₙₛ^{HS} is a frequency-independent weighting factor for the integration of the HS component of the translational VDoS, Iₜᵣₐₙₛ^{HS}(v), to obtain Sₜᵣₐₙₛ^{HS} as described in 2PT theory [Persson et al., J Chem Theory Comput 13:4467-4481 (2017); Lin et al., J Phys Chem B 114:8191-8198 (2010)]. Wₜᵣₐₙₛ^{HS}(v) is a frequency-dependent weighting factor for the integration of the HO component of the translational VDoS, Iₜᵣₐₙₛ^{HO}(v), to obtain Sₜᵣₐₙₛ^{HO} as described in 2PT theory [Lin et al., J Phys Chem B 114:8191-8198 (2010)]. Wᵣₒₜ^{RR} is a frequency-independent weighting factor for the integration of the RR component of the rotational VDoS, Iᵣₒₜ^{HS}(v), to obtain Sᵣₒₜ^{RR} as described in 2PT theory [Lin et al., J Phys Chem B 114:8191-8198 (2010)]. Wᵣₒₜ^{HO}(v) is a frequency-dependent weighting factor for the integration of the HO component of the rotational VDoS, Iᵣₒₜ^{HO}(v), to obtain Sᵣₒₜ^{HO} as described in 2PT theory [Lin et al., J Phys Chem B 114:8191-8198 (2010)]. [symbol] [Cᵥ] is the Fourier transform of the translational velocity time correlation function of either a water or ionic solvent molecule, normalized to obtain the corresponding number of DOF upon integration over all frequencies. The separation of

$$\frac{2}{{k}_{B} T}$$

[symbol]

[C_{ω}] into Iₜᵣₐₙₛ^{HS}(v) and Iₜᵣₐₙₛ^{HO}(v) as indicated in Equation XXV is obtained as described in 2PT theory [Lin et al., J Phys Chem B 114:8191-8198 (2010)]. The separation of

$$\frac{2}{{k}_{B} T}$$

[symbol]

[C_{ω}] into Iᵣₒₜ^{HS}(v) and Iᵣₒₜ^{HO}(v) as indicated in Equation XXVI is obtained as described in 2PT theory [Lin et al., J Phys Chem B 114:8191-8198 (2010)].

The entropy of translational DOF was modeled by diffusive Hard Spheres (HS) and a Harmonic Oscillator model (HO) was employed for vibrational DOF. Rotational DOF in HS were replaced by a rigid rotor model. Note: These expressions describe an interpolation of the thermodynamic properties of high/low entropy liquids/solids, which have shown to perform well for various liquids.

### RESULTS

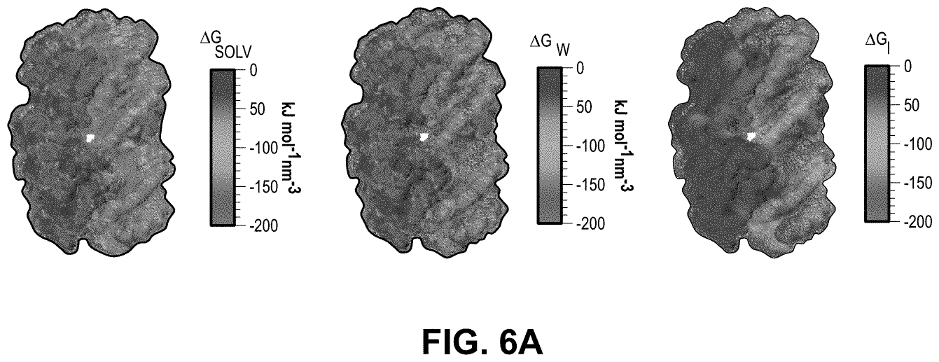

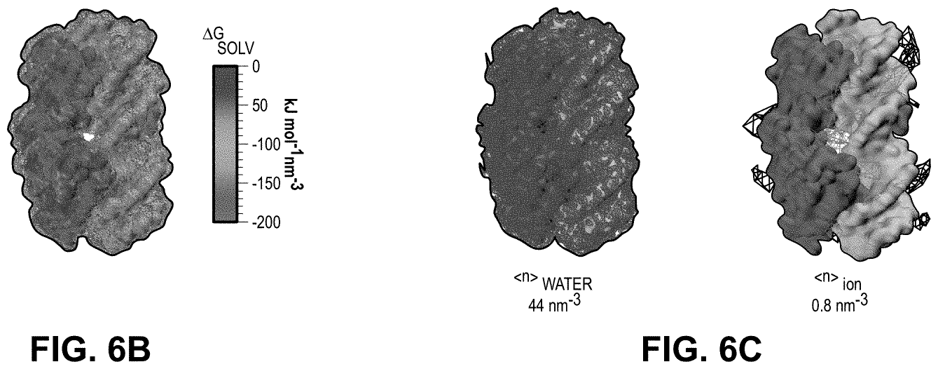

FIGS. 6A-C show a Lambda Repressor N-Terminal domain (NTD)-DNA complex solvated in both a water and electrolytic solvation environment with a negative 39e⁻ charge. In particular, FIG. 6A shows the total solvation free energy and separated contributions by water and ions at the first hydration shell, approx. 3 Å from the solute surface. The solvent environment includes 39 counter charge Na+ ions and 150 mM NaCl. Ions in the solvent preferentially solvate the charged DNA backbone seen in ΔGᵢ. FIG. 6B shows the solvation free energy of the solute resolved in a pure water solvent. Upon spatial integration, ΔGₛₒₗᵥ=−10,686 kJ/mol similar to the solvation free energy contribution by water in the electrolytic system, ΔGₛₒₗᵥ=−10,504 kJ/mol. FIG. 6C shows the solvation sites for water and ion as number densities 1.3 and 20 times greater than bulk for water and ion, respectively.

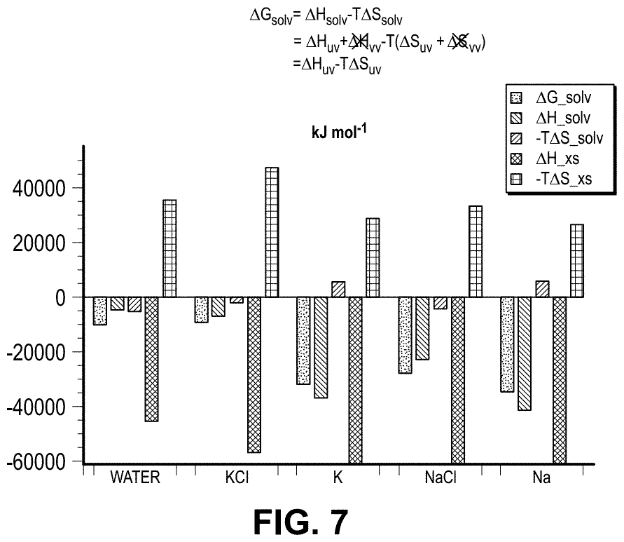

The solvation free energy may be split further into contributions from the solute-solvent and solvent-solvent interactions, which cancel exactly and do not contribute to solvation free energy. FIG. 7 is a plot (kJ/mol (y-axis) and solute complex-solvent (x-axis)) with the spatial integration of the solvation energies shown for the Lambda Repressor-DNA complex in pure water, in Na⁺ and K⁺ charged balance systems, and in 150 mM NaCl and KCl solvents.

The methods described herein are also optionally adapted, for example, to investigate solvation free differences of Hofmeister series ions to find underlying salting out mechanisms or to find mono-ionic solvent cut-off distance convergence where traditional electrostatic screening does not apply, among other applications.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, and/or computer readable media or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

What is claimed is:

1. A method of analyzing solvation free energies and predicting solvent-mediated interactions between solvated molecules using a computer, the method comprising:
defining, by the computer, a plurality of three-dimensional (3D) grids of voxels on at least a portion of two or more simulated target molecules solvated with simulated solvent molecules to produce a 3D simulation structure, wherein the simulated solvent molecules comprise one or more simulated nonionic solvent molecules and one or more simulated ionic solvent molecules, wherein a first 3D grid of the plurality of 3D grids comprises a first spatial resolution, which first 3D grid is defined on at least some of the simulated nonionic solvent molecules, and wherein a second 3D grid of the plurality of 3D grids comprises a second spatial resolution that differs from the first spatial resolution, which second 3D grid is defined on at least some of the simulated ionic solvent molecules;
determining, by the computer, one or more thermodynamic, dynamic, and/or structural parameters using the 3D simulation structure as part of one or more atomistic simulations to produce at least one set of simulation data; and,
generating, by the computer, at least one 3D solvation free energy map from the set of simulation data, thereby analyzing solvation free energies and predicting solvent-mediated interactions between solvated molecules.

2. The method of claim 1, wherein at least two of the simulated target molecules form a complex with one another.

3. The method of claim 1, wherein at least two of the simulated target molecules are separate from one another.

4. The method of claim 1, comprising determining a difference in solvation free energies between when the simulated target molecules form a complex with one another and when the simulated target molecules are separate from one another.

5. The method of claim 1, wherein at least two of the simulated target molecules are identical to one another and/or wherein at least two of the simulated target molecules differ from one another.

6. The method of claim 1, wherein the simulated target molecules comprise a simulated biomolecule, a simulated pharmaceutical molecule, a simulated organic molecule, a simulated inorganic molecule, a portion thereof, or a combination thereof.

7. The method of claim 1, wherein the simulated nonionic solvent molecules comprise simulated water molecules and/or wherein the simulated ionic solvent molecules comprise simulated monoatomic ionic molecules.

8. The method of claim 1, wherein a concentration of the simulated nonionic solvent molecules is higher than a concentration of the simulated ionic solvent molecules in the 3D simulation structure.

9. The method of claim 1, further comprising identifying one or more at least potential binding sites on one or more of the simulated target molecules from the 3D solvation free energy map.

10. The method of claim 1, further comprising determining an impact of solvation on binding affinity between the simulated target molecules from the 3D solvation free energy map.

11. The method of claim 1, wherein a volume of a given simulated nonionic solvent molecule is greater than a volume of a given voxel in the first 3D grid.

12. The method of claim 1, wherein a volume of a given simulated ionic solvent molecule is less than a volume of a given voxel in the second 3D grid.

13. The method of claim 1, wherein the first spatial resolution is higher than the second spatial resolution.

14. The method of claim 1, wherein the first spatial resolution and the second spatial resolution are sufficient to obtain substantially continuous statistics from the set of simulation data.

15. The method of claim 1, wherein the first spatial resolution is about 1 cubic Angstroms (Å³) and/or wherein the second spatial resolution is about 125 cubic Angstroms (Å³).

16. The method of claim 1, comprising determining spatially resolved enthalpy and entropy contributions from the simulated nonionic solvent molecules and from the simulated ionic solvent molecules.

17. The method of claim 1, comprising distinguishing interactions between the simulated target molecules and the simulated nonionic solvent molecules, interactions between the simulated target molecules and the simulated ionic solvent molecules, interactions between the simulated nonionic solvent molecules, interactions between the simulated ionic solvent molecules, and interactions between the simulated nonionic solvent molecules and the simulated ionic solvent molecules from one another.

18. The method of claim 1, comprising sampling interactions between the simulated nonionic solvent molecules and the simulated ionic solvent molecules in both the first and second spatial resolutions.

19. A system, comprising at least one controller that comprises, or is capable of accessing, computer readable media comprising non-transitory computer-executable instructions which, when executed by at least one electronic processor, perform at least:
defining a plurality of three-dimensional (3D) grids of voxels on at least a portion of two or more simulated target molecules solvated with simulated solvent molecules to produce a 3D simulation structure, wherein the simulated solvent molecules comprise one or more simulated nonionic solvent molecules and one or more simulated ionic solvent molecules, wherein a first 3D grid of the plurality of 3D grids comprises a first spatial resolution, which first 3D grid is defined on at least some of the simulated nonionic solvent molecules, and wherein a second 3D grid of the plurality of 3D grids comprises a second spatial resolution that differs from the first spatial resolution, which second 3D grid is defined on at least some of the simulated ionic solvent molecules;
determining one or more thermodynamic, dynamic, and/or structural parameters using the 3D simulation structure as part of one or more atomistic simulations to produce at least one set of simulation data; and,
generating at least one 3D solvation free energy map from the set of simulation data.

20. A computer readable media comprising non-transitory computer-executable instructions which, when executed by at least one electronic processor, perform at least:
defining a plurality of three-dimensional (3D) grids of voxels on at least a portion of two or more simulated target molecules solvated with simulated solvent molecules to produce a 3D simulation structure, wherein the simulated solvent molecules comprise one or more simulated nonionic solvent molecules and one or more simulated ionic solvent molecules, wherein a first 3D grid of the plurality of 3D grids comprises a first spatial resolution, which first 3D grid is defined on at least some of the simulated nonionic solvent molecules, and wherein a second 3D grid of the plurality of 3D grids comprises a second spatial resolution that differs from the first spatial resolution, which second 3D grid is defined on at least some of the simulated ionic solvent molecules;
determining one or more thermodynamic, dynamic, and/or structural parameters using the 3D simulation structure as part of one or more atomistic simulations to produce at least one set of simulation data; and,
generating at least one 3D solvation free energy map from the set of simulation data.
